(19) United States
(12) Patent Application Publication
(10) Pub. No.: US 2020/0289786 A1
(43) Pub. Date: Sep. 17, 2020

(54) MEASUREMENT APPARATUS AND MEASUREMENT SYSTEM

(71) Applicant: KYOCERA Corporation, Kyoto (JP)

(72) Inventors: Yasuhiko OOWADA, Yokosuka-shi, Kanagawa (JP); Asao HIRANO, Shinagawa-ku, Tokyo (JP); Susumu KASHIWASE, Machida-shi, Tokyo (JP); Takeshi HIGUCHI, Yokohama-shi, Kanagawa (JP)

(73) Assignee: KYOCERA Corporation, Kyoto (JP)

(21) Appl. No.: 16/753,555

(22) PCT Filed: Oct. 1, 2018
(86) PCT No.: PCT/JP2018/036722

§ 371 Date: Apr. 3, 2020

(30) Foreign Application Priority Data
Oct. 26, 2017 (JP) 2017-207479
Jun. 7, 2018 (JP) 2018-109734

(51) Int. Cl. A61M 21/02; G01K 7/22; G01K 13/00; G08B 21/04; G08B 3/10

(52) U.S. Cl. CPC A61M 21/02; G01K 7/22; G01K 13/004; G08B 21/0453; A61M 2021/0027; A61M 2230/50; A61M 2210/0662; A61M 2205/8206; G08B 3/10

(57) ABSTRACT

A measurement apparatus includes a wearing portion, a body portion, a first measurement portion, and a second measurement portion. The wearing portion is configured to be worn at an auricle of a subject. The body portion is joined to the wearing portion. The first measurement portion is joined to the body portion, and configured to be worn by the subject and measure oxygen saturation. The second measurement portion is joined to the body portion, and configured to be in contact with the subject in a state in which the wearing portion is worn and measure body temperature.

### CROSS REFERENCE TO RELATED APPLICATION

This application claims priority to and the benefit of Japanese Patent Application No. 2017-207479 filed on Oct. 26, 2017 and Japanese Patent Application No. 2018-109734 filed on Jun. 7, 2018, the entire disclosures of which are incorporated herein by reference.

### TECHNICAL FIELD

The present disclosure relates to a measurement apparatus and a measurement system.

### BACKGROUND

Measurement apparatuses that are worn on the human body to measure biological information are conventionally known. For example, PTL 1 discloses an ear-worn type apparatus that is worn at the ears, detects biological information, and calculates a blood flow state value based on the detected biological information.

### CITATION LIST

### Patent Literature

PTL 1: JP 2005-192581 A

### SUMMARY

A measurement apparatus according to an aspect comprises:

a wearing portion configured to be worn at an auricle of a subject;

a body portion joined to the wearing portion;

a first measurement portion joined to the body portion, and configured to be worn by the subject and measure oxygen saturation; and

a second measurement portion joined to the body portion, and configured to be in contact with the subject in a state in which the wearing portion is worn and measure body temperature.

A measurement apparatus according to another aspect comprises:

a wearing portion configured to be worn at an auricle of a subject;

a body portion including a connector configured to removably connect a measurement instrument capable of measuring biological information of the subject; and

a body temperature measurement portion joined to the body portion, and configured to be in contact with the subject in a state in which the wearing portion is worn and measure body temperature.

A measurement system according to an aspect comprises:

a measurement apparatus including: a wearing portion configured to be worn at an auricle of a subject; a body portion including a connector; and a body temperature measurement portion joined to the body portion, and configured to be in contact with the subject in a state in which the wearing portion is worn and measure body temperature; and

a measurement instrument configured to be removably connected to the connector, and measure biological information of the subject.

### BRIEF DESCRIPTION OF THE DRAWINGS

In the accompanying drawings:

FIG. 1 is a schematic external perspective view of a measurement apparatus according to an embodiment;

FIG. 2 is a schematic external perspective view of the measurement apparatus in FIG. 1 as seen from a different direction;

FIG. 3 is a schematic view illustrating an example of a state of wearing the measurement apparatus in FIG. 1;

FIG. 4 is a schematic external perspective view illustrating the measurement apparatus in the state in which a measurement instrument is connected to a connector;

FIG. 5 is a schematic view illustrating an example of a state of wearing a measurement apparatus according to an embodiment;

FIG. 6 is a functional block diagram illustrating a schematic structure of the measurement apparatus and the measurement instrument in FIG. 4;

FIG. 7 is a flowchart illustrating an example of a process performed by the measurement apparatus in FIG. 6;

FIG. 8 is a flowchart illustrating an example of a process performed by a measurement apparatus according to an embodiment; and

FIG. 9 is a functional block diagram illustrating a schematic structure of a measurement system according to an embodiment.

### DETAILED DESCRIPTION

In the case of measuring biological information using a conventional measurement apparatus, a different measurement apparatus needs to be used depending on which biological information is to be measured. Hence, a subject needs to wear a plurality of measurement apparatuses in sequence according to the order in which biological information is measured, which is often considered inconvenient. The present disclosure relates to provision of a measurement apparatus and a measurement system capable of improving convenience. According to the present disclosure, a measurement apparatus and a measurement system capable of improving convenience can be provided. An embodiment will be described in detail below, with reference to the drawings.

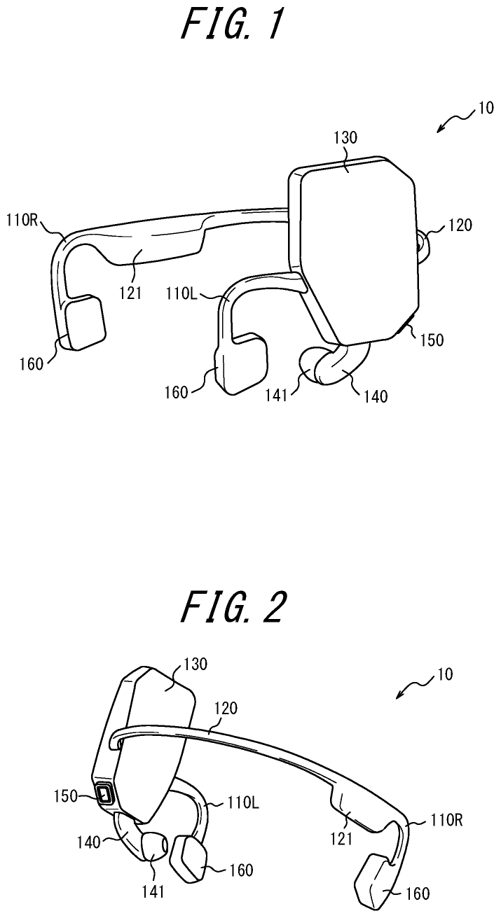

FIG. 1 is a schematic external perspective view of a measurement apparatus 10 according to an embodiment. FIG. 2 is an external perspective view of the measurement apparatus 10 in FIG. 1 as seen from a different direction. That is, FIGS. 1 and 2 are external perspective views of the same measurement apparatus 10 as seem from different directions.

The measurement apparatus 10 is used in a state of being worn by a subject. The measurement apparatus 10 may be worn at any position where biological information of the subject can be measured. Non-limiting examples of the position at which the measurement apparatus 10 is worn include the head, the neck, the arms, the wrists, the abdomen, the shoulders, the waist, the hips, the legs, the ankles, and the fingers and toes. This embodiment describes an example in which the measurement apparatus 10 is worn on the head of the subject. Specifically, the measurement apparatus 10 according to this embodiment is worn at the auricles of the subject.

The measurement apparatus 10 measures biological information of the subject, in a state of being worn at the auricles of the subject. The biological information is any information about the organism, and may include, for example, oxygen saturation, body temperature, pulse rate, breathing rate, perfusion index (PI) value, blood flow amount, and blood pressure. The biological information may also include, for example, the degree of relaxation indicating the degree of physical and mental relaxation of the organism. The measurement apparatus 10 may estimate the state of the subject based on the measured biological information. The state of the subject is any state of the organism of the subject, and includes, for example, the possibility of developing mountain sickness.

The measurement apparatus 10 includes a first wearing portion 110R, a second wearing portion 110L, and a joining portion 120.

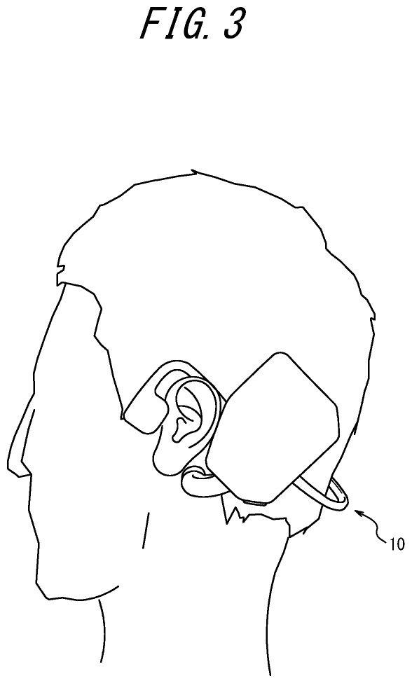

The first wearing portion 110R is worn at the right auricle of the subject. That is, when the subject wears the measurement apparatus 10, the first wearing portion 110R is in contact with the root of the right auricle of the subject on the side closer to the top of the head, and maintains the wearing state of the measurement apparatus 10. The second wearing portion 110L is worn at the left auricle of the subject. That is, when the subject wears the measurement apparatus 10, the second wearing portion 110L is in contact with the root of the left auricle of the subject on the side closer to the top of the head. For example, in the state in which the subject wears the measurement apparatus 10, the measurement apparatus 10 is supported by the first wearing portion 110R and the second wearing portion 110L worn at the auricles, as illustrated in FIG. 3.

The first wearing portion 110R and the second wearing portion 110L may have curved shapes as illustrated in FIGS. 1 and 2 as an example, so as to be easily supported respectively by the right auricle and the left auricle when the subject wears the measurement apparatus 10. The first wearing portion 110R and the second wearing portion 110L may be symmetrically shaped. Herein, the first wearing portion 110R and the second wearing portion 110L are collectively referred to as wearing portions 110 when they are not distinguished from each other.

The joining portion 120 joins the first wearing portion 110R and the second wearing portion 110L. The joining portion 120 has a curved shape, and is configured to be located at the back of the head of the subject in the wearing state of the measurement apparatus 10. The first wearing portion 110R, the second wearing portion 110L, and the joining portion 120 may be symmetrically shaped.

The joining portion 120 may be shaped so as not to interfere with the subject wearing another apparatus on the head. For example, the subject may wear a helmet, glasses, a cap, or the like as another apparatus. The joining portion 120 may be shaped so that the subject can wear a helmet, glasses, a cap, or the like even when he or she wears the measurement apparatus 10. For example, the joining portion 120 may be shaped so as to be located closer to the neck of the subject than the back of the head of the subject in the wearing state of the measurement apparatus 10. The joining portion 120 may be shaped so as to cover the top of the head.

The joining portion 120 is provided with a body portion 130 that includes a substrate for controlling a measurement process by the measurement apparatus 10. In detail, the body portion 130 is joined to the wearing portions 110 via the joining portion 120, and supported by the wearing portions 110 in the wearing state of the measurement apparatus 10. The body portion 130 may have a thin platelike shape. With such a body portion 130, the subject can easily wear the measurement apparatus 10. Moreover, such a body portion 130 is unlikely to cause discomfort to the subject in the wearing state of the measurement apparatus 10.

A body temperature measurement portion 140 is joined to the body portion 130. For example, the body temperature measurement portion 140 may be shaped to project from the body portion 130. The body temperature measurement portion 140 may be joined to the body portion 130 so as to be in contact with the mastoid part of the subject in the state in which the subject wears the measurement apparatus 10. That is, in the wearing state of the measurement apparatus 10, a tip 141 of the projecting body temperature measurement portion 140 is in contact with the mastoid part of the subject. The mastoid part is a part between the auricle and the back of the head. For example, the tip 141 of the projecting body temperature measurement portion 140 includes a thermistor, and the body temperature measurement portion 140 can measure the body temperature of the subject by the thermistor. In this embodiment, the tip 141 of the body temperature measurement portion 140 is configured to be in contact with the left mastoid part of the subject, as illustrated in FIG. 3 as an example. The body temperature measurement portion 140 may be configured to measure the body temperature by detecting infrared light. The body temperature measurement portion 140 may be joined to the body portion 130 so as to be in contact with a part other than the mastoid part of the subject in the state in which the subject wears the measurement apparatus 10. The body temperature measurement portion 140 may measure the body temperature of the subject without being in contact with the subject in the state in which the subject wears the measurement apparatus 10, in the body portion 130.

The body temperature measurement portion 140 may be configured to be biased toward the mastoid part of the subject in the wearing state of the measurement apparatus 10. For example, the body temperature measurement portion 140 may be joined to the body portion 130 via a spring, and configured to be biased toward the mastoid part by the elastic force of the spring. The body temperature measurement portion 140 may be configured to be biased toward the mastoid part by a mechanism other than a spring. The force for biasing the body temperature measurement portion 140 may be, for example, such that the subject wearing the measurement apparatus 10 feels no pain. The force for biasing the body temperature measurement portion 140 may be, for example, such that the tip 141 of the body temperature measurement portion 140 does not separate from the mastoid part.

The body portion 130 includes a connector 150 to which a measurement instrument capable of measuring biological information of the subject is removably connectable. For example, the connector 150 may be formed as a female connector. The connector 150 may have a shape conforming to a predetermined standard. The connector 150 may be connected with, for example, a measurement instrument capable of measuring predetermined biological information depending on the state of the subject to be measured (or estimated). This embodiment describes an example in which the connector 150 is connected with a measurement instrument capable of measuring the oxygen saturation of the subject.

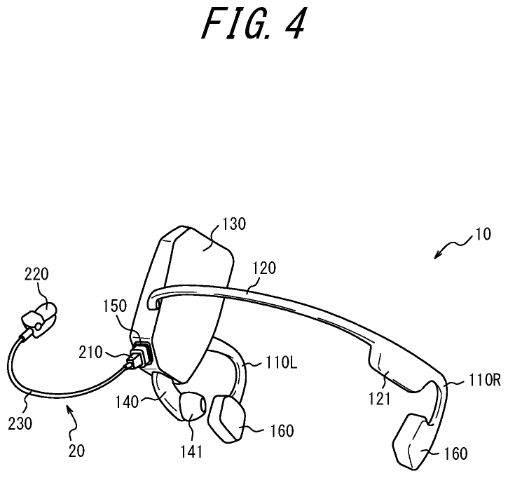

FIG. 4 is a schematic external perspective view illustrating the measurement apparatus 10 in the state in which a measurement instrument 20 is connected to the connector 150. The measurement instrument 20 may be, for example, an instrument having a function corresponding to a pulse oximeter, which is capable of measuring oxygen saturation. For example, the measurement instrument 20 is capable of measuring, as the oxygen saturation of the subject, percutaneous oxygen saturation (SpO₂, S: saturation, p: percutaneous or pulse oximetry, O₂: oxygen). The biological information measured by the measurement instrument 20 is, however, not limited to SpO₂ and blood flow amount. Hereafter, percutaneous oxygen saturation (SpO₂) is also simply referred to as oxygen saturation. One value indicating oxygen saturation is SaO₂ (S: saturation, a: artery, O₂: oxygen), and SaO₂ represents a measured value of the oxygen saturation of arterial blood. By measuring SpO₂, SaO₂ can be measured indirectly. If measurement conditions are right, the two values are approximate to each other.

The measurement instrument 20 in FIG. 4 includes a connector 210 connectable to the connector 150 in the measurement apparatus 10, a measurement portion 220 capable of measuring biological information at a measured part, and a cable 230 joining the connector 210 and the measurement portion 220.

The connector 210 may be formed as, for example, a male connector of a shape removably connectable to the connector 150 in the measurement apparatus 10.

The measurement portion 220 may be configured to, for example, sandwich the measured part to be worn at the measured part. For example, the measurement portion 220 is configured to sandwich the earlobe as the measured part to be worn at the earlobe. For example, the biological information may be measured at, as the measured part, the earlobe of the auricle on the side opposite to the body temperature measurement portion 140. In the case of using the measurement apparatus 10 illustrated in FIGS. 1 to 4, the measurement portion 220 can acquire the biological information from the earlobe of the right ear as the measured part. The measurement portion 220 may acquire the biological information from the earlobe of the left ear as the measured part, or acquire the biological information from the earlobes of both the right and left ears as the measured part.

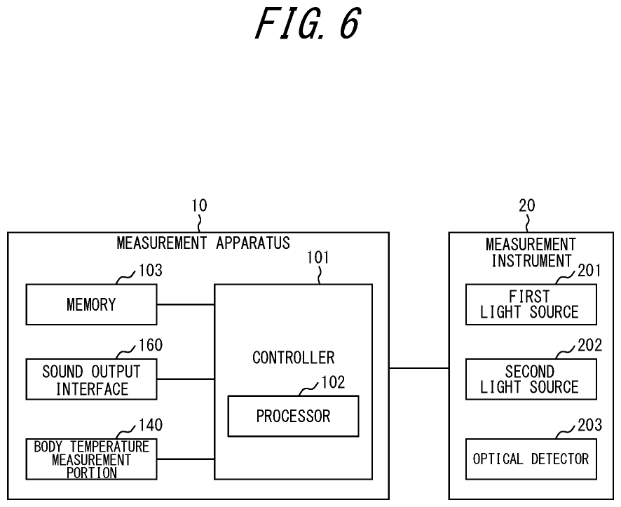

The measurement portion 220 includes two light sources, i.e. a first light source and a second light source, as illustrated in FIG. 6 described later. The first and second light sources emit light of different wavelengths. The first light source emits light of a first wavelength, and the second light source emits light of a second wavelength different from the first wavelength. The first wavelength is a wavelength at which the difference between the absorbance of hemoglobin combined with oxygen (hereafter also referred to as “oxygenated hemoglobin”) and the absorbance of hemoglobin not combined with oxygen (hereafter also referred to as “reduced hemoglobin”) is large. For example, the first wavelength is a wavelength of 600 nm to 700 nm, and the light emitted from the first light source is red light. The second wavelength is a wavelength at which the difference between the absorbance of oxygenated hemoglobin and the absorbance of reduced hemoglobin is small as compared with the first wavelength. For example, the second wavelength is a wavelength of 800 nm to 1000 nm, and the light emitted from the second light source is near-infrared light.

The measurement portion 220 further includes an optical detector capable of receiving light transmitted through the body's tissues (measured part) from among the light emitted from the first and second light sources. The optical detector outputs a signal corresponding to the intensity of the received light, to the body portion 130. The measurement portion 220 may include two optical detectors, i.e. a first optical detector and a second optical detector, respectively capable of receiving the transmitted light of the light emitted from the first and second light sources, or include one optical detector capable of receiving the transmitted light of the light emitted from the first and second light sources. This embodiment describes an example in which the measurement portion 220 includes one optical detector.

In the body portion 130, for example, the below-described controller calculates SpO₂ based on the signal acquired from the optical detector. Specifically, the controller calculates SpO₂ based on the difference in received light intensity in the optical detector between when the first light source irradiates the measured part and when the second light source irradiates the measured part. The controller can further calculate the pulse rate, based on the temporal change in received light intensity in the optical detector. Specifically, the controller can calculate the period of received light intensity from the temporal change in received light intensity, and calculate the pulse rate per unit time based on the period. The controller can further calculate the PI value, based on the temporal change in received light intensity in the optical detector. The PI value is also referred to as “perfusion index”, and is expressed as the proportion of the pulsatile component to the non-pulsatile component in the blood flow. The controller can calculate PI, by calculating the proportion of the pulsatile component to the non-pulsatile component in the blood flow from the temporal change in received light intensity. The controller can further calculate the breathing rate, based on the temporal change in received light intensity in the optical detector. For example, the controller calculates the breathing rate by extracting the low frequency component of the temporal change in received light intensity in the optical detector.

Referring back to FIGS. 1 and 2, the joining portion 120 may include a battery holder 121. The battery holder 121 includes a battery for driving each functional part included in the measurement apparatus 10.

The body portion 130 and the battery may be arranged in the joining portion 120 so that the force exerted from the first wearing portion 110R on the right auricle and the force exerted from the second wearing portion 110L on the left auricle are approximately equal. That is, the body portion 130 and the battery may be arranged in the joining portion 120 so that the right-left weight balance is approximately equal in the wearing state of the measurement apparatus 10. Herein, the phrase “approximately equal” includes a range in which the subject wearing the measurement apparatus 10 feels no discomfort in terms of weight balance. That is, the phrase “approximately equal” includes a range in which the subject does not feel that the right and left weights are not balanced in the wearing state of the measurement apparatus 10. The body portion 130 and the battery may be, for example, arranged at corresponding positions on the left and the right in the joining portion 120. In the example illustrated in FIGS. 1 and 2, the battery is located near the first wearing portion 110R, and the body portion 130 is located near the second wearing portion 110L.

The measurement apparatus 10 according to this embodiment further includes a sound output interface 160 that causes the subject wearing the measurement apparatus 10 to hear sound. In this embodiment, the sound output interface 160 is located on the tip side of the wearing portion 110 not joined to the joining portion 120. In this embodiment, the sound output interface 160 is configured to be located at the harmony crevice or temple of the subject in the wearing state of the measurement apparatus 10. The sound output interface 160 may be formed by, for example, a bone conduction speaker for causing the subject to hear sound by vibration transmission to the human body. In this case, for example, the sound output interface 160 vibrates based on a control signal from the controller in the body portion 130. As a result of the vibration of the sound output interface 160 propagating to the skull of the subject, the subject can hear sound. In this case, the measurement apparatus 10 can make the subject hear sound without covering the ear of the subject, so that the subject can hear ambient sound. The sound output interface 160 may be configured to be located at any position of the body of the subject, such as the temporal region, the forehead, any other part of the head, or the neck, in the wearing state of the measurement apparatus 10.

The sound output interface 160 may not necessarily be formed by a bone conduction speaker. The sound output interface 160 may be formed by an apparatus for transmitting sound to a user by air vibration, such as an earphone or a speaker. In this case, the sound output interface 160 outputs sound based on a control signal from the controller. In the present disclosure, a bone conduction speaker and an apparatus for transmitting sound to a user by air vibration may be used together. That is, the sound output interface 160 may be any combination of a bone conduction speaker and an apparatus for transmitting sound to a user by air vibration.

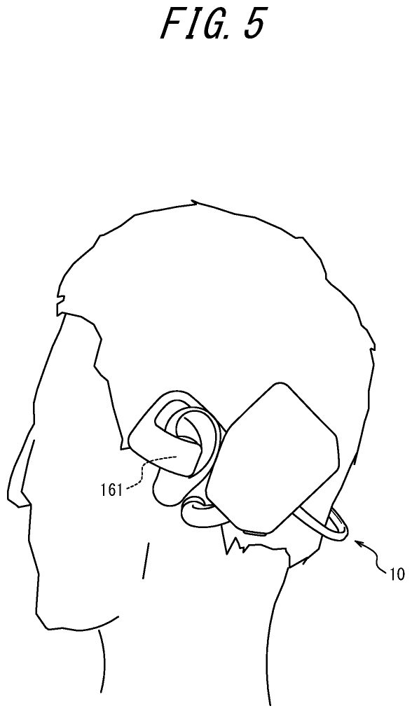

FIG. 5 is a view illustrating the state in which the subject wears the measurement apparatus 10 in the case where the sound output interface 161 is formed by an earphone, a speaker, or the like. The measurement apparatus 10 in FIG. 5 has the same structure as the measurement apparatus 10 in FIG. 3, except that the sound output interface 160 in the measurement apparatus 10 in FIG. 3 has been replaced with a sound output interface 161. In the case where the sound output interface 160 is formed by a bone conduction speaker, there is no need to cover the ear of the subject when causing the subject to hear sound, as described earlier with reference to FIG. 3. In the case where the sound output interface 161 is formed by an earphone, a speaker, or the like, on the other hand, the portion of the sound output interface 161 from which sound is output may be close to the auricle or external ear canal hole of the subject, as illustrated in FIG. 5. For example, in the case where the sound output interface 161 is formed by a speaker, the portion of the sound output interface 161 from which sound is output may be configured to abut on any part of the auricle of the subject (e.g. near the external ear canal hole). For example, in the case where the sound output interface 161 is formed by an earphone, at least part of the portion (e.g. earpiece) of the sound output interface 161 from which sound is output may be configured to be inserted in the external ear canal hole of the subject. In the case where the sound output interface 161 is formed by an earphone, a speaker, or the like as illustrated in FIG. 5, the subject may find it harder to hear ambient sound, but can easily concentrate on the sound output from the sound output interface 161.

In the example illustrated in FIGS. 1 and 2, the sound output interface 160 is provided on the tip side of each of the first wearing portion 110R and the second wearing portion 110L. The sound output interface 161, on the other hand, may be provided on the tip side of only one of the first wearing portion 110R and the second wearing portion 110L, or provided at any position of the measurement apparatus 10 other than the tip side of the wearing portion. In the following description, the sound output interface 160 is replaceable with the sound output interface 161 as appropriate.

The measurement apparatus 10 according to an embodiment can perform measurement by at least one of the measurement instrument 20 and the body temperature measurement portion 140 while the sound output interface 160 is outputting predetermined sound. In the measurement apparatus 10 according to an embodiment, the sound output interface 160 can output predetermined sound based on information measured by at least one of the measurement instrument 20 and the body temperature measurement portion 140. In the measurement apparatus 10 according to an embodiment, the sound output by the sound output interface 160 may be sound having a psychological and/or physiological action on the subject using the measurement apparatus 10. For example, in the measurement apparatus 10 according to an embodiment, the sound output by the sound output interface 160 may be sound including solfeggio frequencies.

Herein, the solfeggio frequencies are sound frequencies considered to have some kind of effect on the human mind and/or body. For example, the following are known as sounds of frequencies considered to have effect on the human mind and/or body based on past studies. Sound of 174 Hz is considered to be effective in pain alleviation. Sound of 285 Hz is considered to be effective in expansion and promotion of consciousness from multidimensional domain. Sound of 396 Hz is considered to be effective in release from guilt, trauma, and fear. Sound of 417 Hz is considered to be effective in recovery from negativity and promotion of transformation. Sound of 528 Hz is considered to be effective in conversion to ideals, miracle, and cell recovery. Sound of 639 Hz is considered to be effective in repair of human connections and relationships. Sound of 741 Hz is considered to be effective in expressiveness improvement and problem solution. Sound of 852 Hz is considered to be effective in raising intuition and awareness. Sound of 963 Hz is considered to be effective in connection with high-dimensional, cosmic consciousness. For example, while the sound output interface 160 is outputting sound of 396 Hz, the measurement apparatus 10 can perform measurement by at least one of the measurement instrument 20 and the body temperature measurement portion 140 on the subject who is in a relaxed state.

In the measurement apparatus 10 according to an embodiment, the sound output by the sound output interface 160 may be, for example, sound constituting music (typically, sound including melody), or predetermined sound effects such as environmental sound (typically, sound not including melody). The predetermined sound effects such as environmental sound may include, for example, sound of leaves rustling in the wind with occasional sound of chirping birds, which conjure images of forests. The predetermined sound effects such as environmental sound may include, for example, sound of waves, which conjures images of beaches. In the measurement apparatus 10 according to an embodiment, the sound output by the sound output interface 160 may be, for example, a mixture of sound constituting music (typically, sound including melody) and predetermined sound effects such as environmental sound (typically, sound not including melody).

In the measurement apparatus 10 according to an embodiment, the sound output by the sound output interface 160 may be sound having a favorable action on the subject using the measurement apparatus 10, such as the subject's favorite music. While the sound output interface 160 is outputting such sound, the measurement apparatus 10 can perform measurement by at least one of the measurement instrument 20 and the body temperature measurement portion 140 on the subject who is in a favorable state.

FIG. 6 is a functional block diagram illustrating a schematic structure of the measurement apparatus 10 and the measurement instrument 20 in FIG. 4. The measurement apparatus 10 includes a controller 101, a memory 103, the body temperature measurement portion 140, and the sound output interface 160 as functional parts, as illustrated in FIG. 6.

The controller 101 includes at least one processor 102 that controls and manages the whole measurement apparatus 10, e.g. each functional block in the measurement apparatus 10 and the measurement instrument 20. The controller 101 includes at least one processor 102 such as a CPU (Central Processing Unit) that executes a program defining a control procedure, to achieve its functions. Such a program is, for example, stored in the memory 103 or an external storage medium connected to the measurement apparatus 10.

In various embodiments, at least one processor 102 may be implemented as a single integrated circuit (IC), or as a plurality of ICs and/or discrete circuits communicably connected to one another. At least one processor 102 can be implemented according to various known technologies.

In one embodiment, the processor 102 includes, for example, one or more circuits or units configured to perform one or more data calculation procedures or processes by executing instructions stored in related memory. In another embodiment, the processor 102 may be firmware (e.g. a discrete logic component) configured to perform one or more data calculation procedures or processes.

In various embodiments, the processor 102 may include one or more processors, controllers, microprocessors, microcontrollers, application specific integrated circuits (ASICs), digital signal processors, programmable logic devices, field programmable gate arrays, or any combination of these devices or structures, or any combination of other known devices or structures, to perform the functions of the controller 101 described below.

The controller 101 controls biological information measurement processes. For example, the controller 101 controls a process of measuring the body temperature of the subject by the body temperature measurement portion 140. For example, the controller 101 controls a process of measuring the SpO₂ of the subject by the measurement instrument 20. The controller 101 may simultaneously perform the body temperature measurement process by the body temperature measurement portion 140 and the SpO₂ measurement process by the measurement instrument 20. The controller 101 can thus measure the body temperature and SpO₂ at the same time.

The controller 101 may transmit a control signal to the sound output interface 160 to cause the subject to hear sound such as music described above, while performing the body temperature measurement process by the body temperature measurement portion 140 and the SpO₂ measurement process by the measurement instrument 20. This helps the subject relax, and not be conscious of the fact that biological information is being measured. Consequently, the measurement apparatus 10 can measure more accurate biological information.

The controller 101 may estimate the state of the subject based on the measured information. In this embodiment, for example, the controller 101 may estimate the possibility of the subject developing mountain sickness (also referred to as altitude sickness), based on the measured body temperature and SpO₂ of the subject. Mountain sickness tends to be developed as a result of a decrease in SpO₂. Moreover, with mountain sickness, the body temperature is higher than in normal time. The controller 101 can therefore estimate the possibility of the subject developing mountain sickness, based on body temperature and SpO₂. The controller 101 may estimate the possibility of developing mountain sickness by, for example, weighing body temperature and SpO₂ using a predetermined algorithm. Conventionally, in the case of measuring body temperature and SpO₂ by, for example, wearing a thermometer and a pulse oximeter in sequence, it is impossible to measure body temperature and SpO₂ at the same time. It is therefore difficult to estimate the possibility of developing mountain sickness based on two types of information, i.e. body temperature and SpO₂. With the measurement apparatus 10 according to this embodiment, however, body temperature and SpO₂ can be measured simultaneously, so that the possibility of developing mountain sickness can be estimated based on two indices, i.e. body temperature and SpO₂. The measurement apparatus 10 according to this embodiment thus achieves higher estimation accuracy than in the case of estimating the possibility of developing mountain sickness based on any one index.

The controller 101 may notify the subject of the measured biological information and/or the information about the estimated possibility of developing mountain sickness via the sound output interface 160, by controlling the sound output interface 160. This allows the subject to learn the information. For example, in the case where the subject is notified that his or her possibility of developing mountain sickness is high, the subject can take measures to prevent mountain sickness beforehand.

The memory 103 may be formed by semiconductor memory, magnetic memory, or the like. The memory 103 stores various information, programs for operating the measurement apparatus 10, and the like. The memory 103 may function as working memory. For example, the memory 103 may store the body temperature and SpO₂ of the subject calculated by the controller 101, as historical information. The memory 103 may store information about the possibility of developing mountain sickness estimated by the controller 101.

In an embodiment, the memory 103 may store information of the sound output by the sound output interface 160. The information of the sound stored in the memory 103 may be, for example, an audio file in any format such as an MP3 (MPEG-1 Audio Layer-3) file or a WAV file. The information of the sound stored in the memory 103 may be, for example, any data that can be supplied to each type of synthesizer or sequencer, such as an MIDI (Musical Instrument Digital Interface) data. In an embodiment, the memory 103 may store information of each type of sound depending on the state of the subject using the measurement apparatus 10.

The structures and functions of the body temperature measurement portion 140 and the sound output interface 160 are as described above, and accordingly their detailed description is omitted here.

The measurement instrument 20 includes, in the measurement portion 220, a first light source 201, a second light source 202, and an optical detector 203 as functional parts. The structures and functions of the first light source 201, the second light source 202, and the optical detector 203 are as described above, and accordingly their detailed description is omitted here. The measurement instrument 20 has a function corresponding to a pulse oximeter, and measures the SpO₂ of the subject.

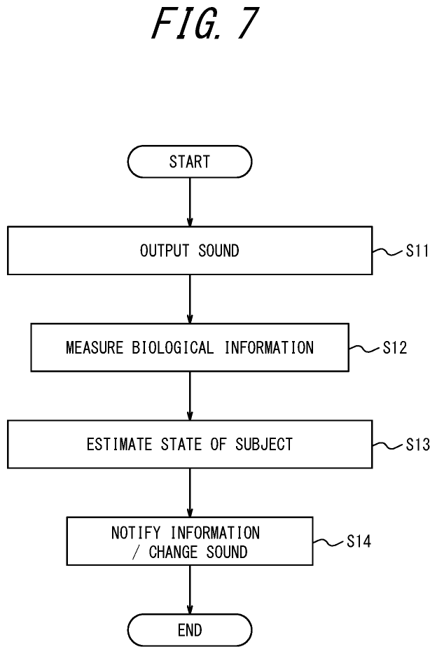

FIG. 7 is a flowchart illustrating an example of a process performed by the measurement apparatus 10. For example, when the subject connects the measurement instrument 20 to the measurement apparatus 10, wears the measurement apparatus 10, and performs an input operation for executing the measurement process, the measurement apparatus 10 starts the process in the flowchart in FIG. 7.

First, the controller 101 in the measurement apparatus 10 controls the sound output interface 160 to output sound (step S11). In step S11, the controller 101 transmits a control signal for outputting predetermined sound, to the sound output interface 160. Consequently, for example in the case where the sound output interface 160 is formed by a bone conduction speaker, the sound output interface 160 vibrates to cause the subject to hear the sound. The sound output from the sound output interface 160 in step S11 may be, for example, such sound that puts the subject in a relaxed state.

The measurement apparatus 10 then measures biological information (step S12). In this embodiment, specifically, the measurement apparatus 10 measures the body temperature of the subject by the body temperature measurement portion 140, and measures the SpO₂ of the subject by the measurement instrument 20. Information about the measured body temperature and SpO₂ is transmitted to the controller 101. Thus, the measurement apparatus 10 according to an embodiment may perform measurement by at least one of the body temperature measurement portion 140 and the measurement instrument 20 while the sound output interface 160 is outputting the predetermined sound. In step S12, the measurement apparatus 10 according to an embodiment may store the result of the measurement by at least one of the body temperature measurement portion 140 and the measurement instrument 20 in, for example, the memory 103.

By accumulating measurement results obtained while causing the subject using the measurement apparatus 10 to hear various sounds in the memory 103, the measurement apparatus 10 may determine a tendency of specific sound influencing a specific subject. For example, the measurement apparatus 10 may determine that, when a specific subject is caused to hear specific music, the subject tends to relax, from past information accumulated in the memory 103. This suggests that, for example in the case where the specific subject is estimated to be under stress, the measurement apparatus 10 may be able to effectively alleviate the stress of the subject by causing the specific subject to hear the specific music. The measurement apparatus 10 according to an embodiment can thus perform measurement on the subject in a relaxed, natural state by causing the subject to hear the predetermined sound and relax. Therefore, the measurement apparatus 10 according to an embodiment can appropriately measure the biological information of the subject.

The controller 101 in the measurement apparatus 10 estimates the state of the subject based on the measured biological information (step S13). In this embodiment, specifically, the controller 101 estimates the possibility of the subject developing mountain sickness based on the body temperature and SpO₂ of the subject.

In step S13, the controller 101 in the measurement apparatus 10 according to an embodiment may estimate the state of the subject, based on information measured by at least one of the body temperature measurement portion 140 and the measurement instrument 20. For example, the controller 101 may estimate that the possibility of the subject developing mountain sickness is high, in the case where a predetermined condition, such as both of the body temperature and SpO₂ of the subject exceeding predetermined thresholds, is satisfied. For example, the controller 101 may estimate that the subject is in a predetermined healthy state, in the case where the body temperature of the subject is within a first predetermined range and the SpO₂ of the subject is within a second predetermined range.

In step S13, the controller 101 in the measurement apparatus 10 according to an embodiment may estimate the state of the subject, based on information measured by at least one of the body temperature measurement portion 140 and the measurement instrument 20 and satisfying a predetermined condition. For example, only in the case where the measurement result of the body temperature measurement portion 140 and/or the measurement instrument 20 is within a predetermined range, the controller 101 in the measurement apparatus 10 according to an embodiment may use the measurement result in the estimation of the state of the subject. In an embodiment, in the case where the measurement result of the body temperature measurement portion 140 and/or the measurement instrument 20 is outside the predetermined range, the controller 101 may not use the measurement result in the estimation of the state of the subject. For example, suppose it is estimated beforehand that the subject is in a predetermined state if the measurement result of the body temperature measurement portion 140 and/or the measurement instrument 20 is within the predetermined range. In this case, the state of the subject may be estimated using only the measurement result of the body temperature measurement portion 140 and/or the measurement instrument 20 that is within the predetermined range. In particular, a high body temperature range exceeding the body temperature of a specific subject in a healthy state, a body temperature range in which the specific subject is developing hypothermia, and the like may be determined beforehand.

The controller 101 in the measurement apparatus 10 according to an embodiment may take into account at least one of time information and position information when estimating the state of the subject in step S13. The time information may be supplied from outside the measurement apparatus 10, or be information of the time determined by the controller 101. The position information may be supplied from outside the measurement apparatus 10, or supplied from a position information acquisition device such as the GPS included in the measurement apparatus 10. For example, in the case where the subject is determined to be in a state of running, e.g. jogging, from the position information, the controller 101 may estimate the state of the subject by taking into account the exercise state of the subject. For example, in the case where the subject is determined to be in a state of being still from the position information, the controller 101 may estimate the state of the subject by taking into account that the subject is not exercising. Further, the controller 101 may estimate the state of the subject, for example, by taking into account which of morning, noon, night, and midnight the current time is, from the time information. By taking into account other information in this way, the state of the subject can be estimated more accurately.

The controller 101 transmits a control signal to the sound output interface 160, to notify the subject of information via the sound output interface 160 (step S14). For example, the controller 101 notifies the subject of the information by causing the subject to hear predetermined alarm sound or voice.

Instead of or in addition to step S14, the controller 101 in the measurement apparatus 10 according to an embodiment may change the sound output by the sound output interface 160 depending on the state of the subject estimated in step S13. For example, in the case where the subject is estimated to be under stress in step S13, the controller 101 may output such sound that alleviates the stress of the subject in step S14. For example, in the case where the body temperature of the subject is estimated to be in a slightly low state in step S13, the controller 101 may output such sound that lifts the subject's mood in step S14. For example, the state in which the body temperature of the subject is slightly low may be a state in which the body temperature is 1% lower than the average body temperature of the subject or a healthy person or a state in which the body temperature is 2% lower than the average body temperature of the subject or a healthy person. As the state in which the body temperature of the subject is slightly low, the proportion in which the body temperature is lower than the average body temperature of the subject or a healthy person is not limited to 1% or 2%, and may be any value.

Thus, the controller 101 in the measurement apparatus 10 according to an embodiment may change the sound output by the sound output interface 160, based on information measured by at least one of the body temperature measurement portion 140 and the measurement instrument 20. With the measurement apparatus 10 according to an embodiment, for example, causing the subject to hear the predetermined sound when the result of measuring the subject is deteriorating is expected to be effective in prevention of the physical condition of the subject from worsening or in recovery in an initial stage.

The measurement apparatus 10 may continuously cause the subject to hear the predetermined sound via the sound output interface 160 at least during step S12.

The measurement apparatus 10 may repeat steps S12 and S13 or steps S12, S13, and S14 periodically, non-periodically, or continuously. The measurement apparatus 10 can thus successively acquire a history of the biological information of the subject and the state of the subject.

In step S14, the measurement apparatus 10 may notify the subject of the information by a means other than the sound output interface 160. For example, the measurement apparatus 10 may include a vibrator, and notify the subject of the information by vibrating the vibrator. For example, the measurement apparatus 10 may include a display, and notify the subject of the information by displaying the information on the display. The measurement apparatus 10 may notify the subject of the information by any other means that can be recognized by the subject.

Thus, the measurement apparatus 10 according to this embodiment can simultaneously measure a plurality of types of biological information by the body temperature measurement portion 140 included in the measurement apparatus 10 and the measurement instrument 20 connected to the connector 150. In the embodiment described above, the measurement apparatus 10 can simultaneously measure the body temperature and SpO₂ of the subject. With the measurement apparatus 10 according to this embodiment, a plurality of types of biological information can be simultaneously measured using one apparatus, so that convenience can be improved. Moreover, at least one of the body temperature measurement portion 140 included in the measurement apparatus 10 and the measurement instrument 20 connected to the connector 150 may perform measurement while the sound output interface 160 is outputting predetermined sound in the measurement apparatus 10. The measurement apparatus 10 according to this embodiment can thus serve to measure the biological information of the subject appropriately and accurately and also maintain or restore the healthy state of the subject.

The measurement apparatus 10 also estimates the state of the subject using the plurality of types of biological information measured simultaneously. With such a measurement apparatus 10, the accuracy of estimating the state of the subject is improved as compared with the case where the state of the subject is estimated based on one type of biological information or the case where the state of the subject is estimated based on the plurality of types of biological information measured at different timings.

The measurement apparatus 10 is worn on the head, so that both hands of the subject are free in the wearing state of the measurement apparatus 10. Since the subject can use both hands even when wearing the measurement apparatus 10, the safety of the subject is easily ensured. The subject can work using both hands while wearing the measurement apparatus 10.

The foregoing embodiment describes the case where the body portion 130 includes the connector 150 and the measurement instrument 20 capable of measuring SpO₂ is removably connectable to the connector 150. Alternatively, the measurement apparatus 10 may include, for example, a functional part capable of measuring SpO₂ and corresponding to the measurement instrument 20. That is, the measurement apparatus 10 may include the functional part capable of measuring SpO₂ and corresponding to the measurement instrument 20, in a form of being not removably connectable. Even in this case, the measurement apparatus 10 can simultaneously acquire the plurality of types of biological information of the subject by the functional part capable of measuring SpO₂, which functions as a first measurement portion, and the body temperature measurement portion 140 capable of measuring body temperature, which functions as a second measurement portion. Therefore, in this case, too, the measurement apparatus 10 achieves the same effects as above.

The foregoing embodiment describes the case where the body temperature measurement portion 140 is joined to the body portion 130. Alternatively, the body temperature measurement portion 140 may not be joined to the body portion 130. For example, the body portion 130 may further include another connector different from the connector 150, and a body temperature measurement instrument having the same function as the body temperature measurement portion 140 may be removably connectable to the other connector. In this case, too, the measurement apparatus 10 achieves the same effects as above.

The foregoing embodiment describes the case where the body portion 130 is located near the second wearing portion 110L, but the position of the body portion 130 is not limited to such. The body portion 130 can be located at any position where the body portion 130 does not interfere with biological information measurement, in the joining portion 120.

The foregoing embodiment describes the case where the body temperature measurement portion 140 measures the body temperature at the mastoid part of the subject, but the part at which the body temperature is measured is not limited to the mastoid part. The body temperature may be measured at other parts such as the back of the neck or the forehead. The body temperature measurement portion 140 may measure the body temperature at a part where the body temperature of the subject can be accurately measured. The part where the body temperature can be accurately measured is, for example, a part that is less affected by external air.

The foregoing embodiment describes the case where the measurement apparatus 10 measures body temperature and SpO₂ as biological information, but the biological information measured by the measurement apparatus 10 is not limited to such. The measurement apparatus 10 may be configured to acquire necessary biological information as appropriate depending on the test object of the subject. Hence, for example, the measurement apparatus 10 need not necessarily include the body temperature measurement portion 140. In the case of not measuring the body temperature of the subject, the body temperature measurement portion 140 may be omitted from the measurement apparatus 10.

The foregoing embodiment describes the case where the measurement apparatus 10 includes two wearing portions, i.e. the first wearing portion 110R and the second wearing portion 110L, but the measurement apparatus 10 need not necessarily include two wearing portions. For example, the measurement apparatus 10 may include only one wearing portion 110. In this case, the subject uses the measurement apparatus 10, for example with the wearing portion 110 being worn at one auricle. The measurement apparatus 10 is supported by one ear in this case.

The foregoing embodiment describes the case where the measurement apparatus 10 includes the sound output interface 160, but the measurement apparatus 10 need not necessarily include the sound output interface 160. Even without the sound output interface 160, the measurement apparatus 10 can measure biological information as long as it includes a measurement portion for measuring biological information.

The measurement apparatus 10 according to the foregoing embodiment outputs sound from the sound output interface 160 (step S11) before measuring biological information (step S12), as illustrated in FIG. 7. Alternatively, the measurement apparatus 10 according to an embodiment may start measurement of biological information in a state in which sound is not output from the sound output interface 160.

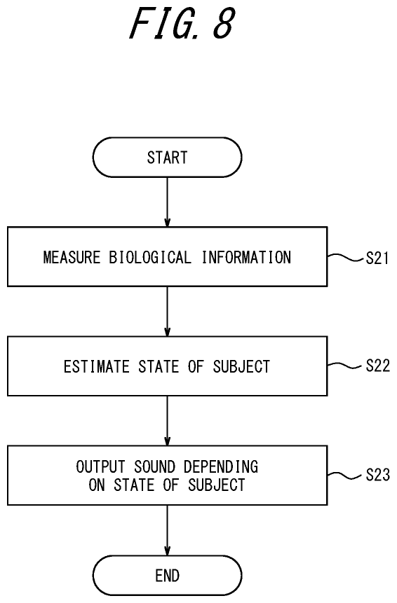

FIG. 8 is a flowchart illustrating an example of a process performed by the measurement apparatus 10 according to an embodiment. For example, when the subject connects the measurement instrument 20 to the measurement apparatus 10, wears the measurement apparatus 10, and performs an input operation for executing the measurement process, the measurement apparatus 10 according to an embodiment starts the process in the flowchart in FIG. 8, as in the embodiment in FIG. 7. In the following, the same description as that with reference to FIG. 7 is simplified or omitted as appropriate.

When the process illustrated in FIG. 8 starts, the controller 101 in the measurement apparatus 10 first measures biological information as in step 12 in FIG. 7 (step S21). The controller 101 then estimates the state of the subject as in step 13 in FIG. 7 (step S22).

After the state of the subject is estimated in step S22, the controller 101 controls the sound output interface 160 to output predetermined sound corresponding to the estimated state of the subject (step S23). For example, in the case where the subject is estimated to be under stress in step S22, the controller 101 may output such sound that alleviates the stress of the subject in step S23, as in step S14 in FIG. 7. For example, in the case where the body temperature of the subject is estimated to be in a slightly low state in step S22, the controller 101 may output such sound that lifts the subject's mood in step S23, as in step S14 in FIG. 7.

Thus, in an embodiment, the sound output interface 160 may output predetermined sound depending on the state of the subject. In this case, the controller 101 may select the predetermined sound output by the sound output interface 160, depending on the estimated state of the subject. Moreover, the memory 103 may store information of the predetermined sound output by the sound output interface 160 depending on the state of the subject. The controller 101 may then select the predetermined sound output by the sound output interface 160 depending on the state of the subject, and read the information of the predetermined sound from the memory 103. With such a measurement apparatus 10 according to an embodiment, too, for example, causing the subject to hear the predetermined sound when the result of measuring the subject is deteriorating is expected to be effective in prevention of the physical condition of the subject from worsening or in recovery in an initial stage.

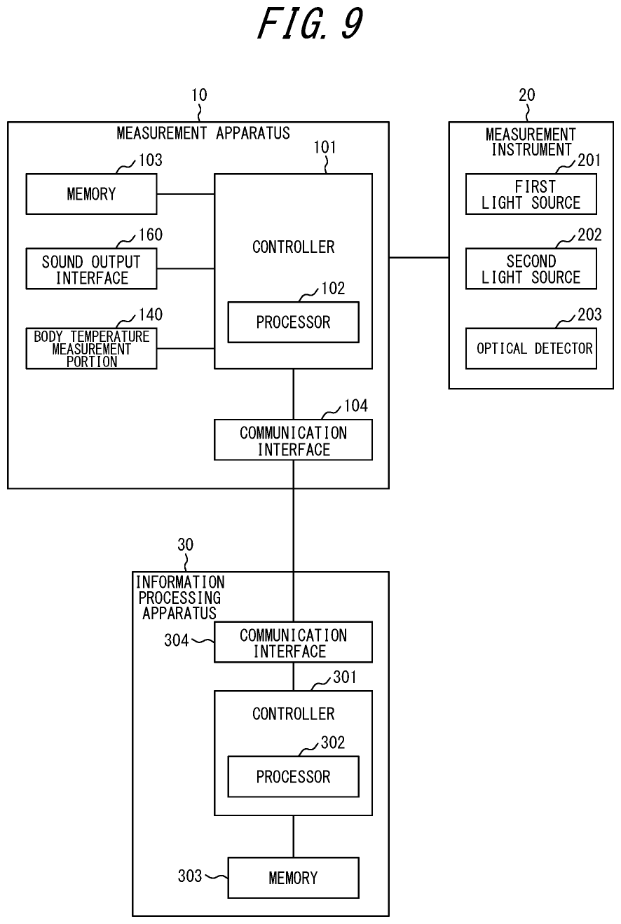

The measurement apparatus 10 according to the foregoing embodiment may be connected to another information processing apparatus in an information communicable state. FIG. 9 is a functional block diagram illustrating a schematic structure of a measurement system 1 according to an embodiment. The measurement system 1 in FIG. 9 includes the measurement apparatus 10, the measurement instrument 20 connected to the measurement apparatus 10, and an information processing apparatus 30. The measurement apparatus 10 and the information processing apparatus 30 are connected so as to be communicable with each other.

In the example illustrated in FIG. 9, the measurement apparatus 10 includes the controller 101, the memory 103, a communication interface 104, the body temperature measurement portion 140, and the sound output interface 160. The structures and functions of the controller 101, the memory 103, the body temperature measurement portion 140, and the sound output interface 160 are as described above, and accordingly their detailed description is omitted here.

The communication interface 104 transmits and receives various information through communication with the information processing apparatus 30. The communication interface 104 can perform information transmission and reception using a wireless network, a wire network, or a combination thereof. The communication interface 104 can perform communication using, for example, Bluetooth® (Bluetooth is a registered trademark in Japan, other countries, or both), infrared, NFC, wireless LAN, wire LAN, or any other communication medium, or any combination thereof. For example, the communication interface 104 transmits biological information measured by the measurement apparatus 10 and/or information about the state of the subject estimated by the measurement apparatus 10, to the information processing apparatus 30.

The functional blocks included in the measurement instrument 20 are the same as those described in the foregoing embodiment, and accordingly their detailed description is omitted here.

The information processing apparatus 30 is formed, for example, by a computer. The information processing apparatus 30 can acquire various information from the measurement apparatus 10, store the acquired information, and perform information processing based on the acquired information. The information processing apparatus 30 includes a controller 301, a memory 303, and a communication interface 304.

The controller 301 includes at least one processor 302 that controls and manages the whole information processing apparatus 30, e.g. each functional block in the information processing apparatus 30. The controller 301 includes at least one processor 302 such as a CPU that executes a program defining a control procedure, to achieve its functions. Such a program is, for example, stored in the memory 303 or an external storage medium connected to the information processing apparatus 30. Specific structures of the processor 302 that can be used include those described with regard to the processor 102.

The memory 303 may be formed by semiconductor memory, magnetic memory, or the like. The memory 303 stores various information, programs for operating the information processing apparatus 30, and the like. The memory 303 may function as working memory. The memory 303 may store information acquired from the measurement apparatus 10.

The communication interface 304 transmits and receives various information through communication with the measurement apparatus 10.

The communication interface 304 can perform information transmission and reception using a wireless network, a wire network, or a combination thereof. The communication interface 304 can perform communication using, for example, Bluetooth® (Bluetooth is a registered trademark in Japan, other countries, or both), infrared, NFC, wireless LAN, wire LAN, or any other communication medium, or any combination thereof. For example, the communication interface 304 receives biological information measured by the measurement apparatus 10 and/or information about the state of the subject estimated by the measurement apparatus 10, from the measurement apparatus 10.

In the case where the presently disclosed techniques are implemented as the measurement system 1 as illustrated in FIG. 9, for example, the measurement apparatus 10 can transmit biological information measured by the measurement apparatus 10 and/or information about the state of the subject estimated by the measurement apparatus 10, to the information processing apparatus 30. For example, the measurement apparatus 10 can transmit information about the body temperature and SpO₂ of the subject and/or information about the possibility of the subject developing mountain sickness, to the information processing apparatus 30. Having received the measured biological information and/or the information about the state of the subject estimated by the measurement apparatus 10, the information processing apparatus 30 may store the received information in the memory 303. Here, the information processing apparatus 30 may store the information received from the measurement apparatus 10 in the memory 303, in association with information identifying the measurement apparatus 10 that has transmitted the information. In this way, the information processing apparatus 30 can accumulate information of a plurality of measurement apparatuses 10.

In the case where the presently disclosed techniques are implemented as the measurement system 1 as illustrated in FIG. 9, for example, the information processing apparatus 30 can perform at least part of the processes performed by the measurement apparatus 10 in the foregoing embodiment. For example, the measurement apparatus 10 performs the processes in steps S11 and 12 in FIG. 7. The measurement apparatus 10 transmits biological information measured in step S12, to the information processing apparatus 30. The information processing apparatus 30 can then perform the process of estimating the state of the subject in step S13 in FIG. 7, based on the information acquired from the measurement apparatus 10. The information processing apparatus 30 transmits information about the estimated state of the subject to the measurement apparatus 10. The measurement apparatus 10 can notify the subject of information, based on the state of the subject estimated by the information processing apparatus 30. In this case, since part of the processes in FIG. 7 is performed by the information processing apparatus 30, the processing load of the measurement apparatus 10 can be reduced.

Although the above describes the case where the measurement portion 220 is configured to, for example, sandwich the measured part to be worn at the measured part, the structure of the measurement portion 220 is not limited to such. For example, the measurement portion 220 may be configured to be worn on the arm, the leg, the head, the wrist, the ankle, or the like by a fixture such as a band, a seal, a bandage, an adhesive, or a fixation mechanism, and irradiate blood vessels in the arm, the leg, the head, the wrist, the ankle, or the like with light to measure SpO₂ or blood flow amount.

### REFERENCE SIGNS LIST

1 measurement system

10 measurement apparatus

20 measurement instrument

30 information processing apparatus

101, 301 controller

102, 302 processor

103, 303 memory

104, 304 communication interface

110 wearing portion

110R first wearing portion

110L second wearing portion

120 joining portion

121 battery holder

130 body portion

140 body temperature measurement portion

141 tip

150 connector

160, 161 sound output interface

201 first light source

202 second light source

203 optical detector

210 connector

220 measurement portion

230 cable

1. A measurement apparatus comprising:
a wearing portion configured to be worn at an auricle of a subject;
a body portion joined to the wearing portion;
a first measurement portion joined to the body portion, and configured to be worn by the subject and measure oxygen saturation; and
a second measurement portion joined to the body portion, and configured to be in contact with the subject in a state in which the wearing portion is worn and measure body temperature.

2. The measurement apparatus according to claim 1, comprising:
an other wearing portion configured to be worn at an other auricle of the subject; and
a joining portion joining the wearing portion and the other wearing portion,
wherein the body portion is located in the joining portion.

3. The measurement apparatus according to claim 2, comprising
a battery configured to drive the measurement apparatus, in the joining portion,
wherein the body portion and the battery are arranged in the joining portion so that a force exerted from the wearing portion on the auricle and a force exerted from the other wearing portion on the other auricle are approximately equal.

4. The measurement apparatus according to claim 1, comprising
a sound output interface configured to cause the subject to hear sound.

5. The measurement apparatus according to claim 4, wherein the sound output interface is configured to cause the subject to hear the sound by vibration transmission to a human body.

6. The measurement apparatus according to claim 4, wherein the sound output interface is formed by an earphone.

7. The measurement apparatus according to claim 4, wherein the sound output interface is configured to output sound having a psychological and/or physiological action on the subject.

8. The measurement apparatus according to claim 7, wherein the sound output interface is configured to output sound including a solfeggio frequency.

9. The measurement apparatus according to claim 4 wherein the sound output interface is configured to output predetermined sound depending on a state of the subject.

10. The measurement apparatus according to claim 9, configured to select the predetermined sound output by the sound output interface, depending on the state of the subject.

11. The measurement apparatus according to claim 10, configured to store information of the predetermined sound output by the sound output interface depending on the state of the subject.

12. The measurement apparatus according to claim 1, configured to estimate a state of the subject based on information measured by at least one of the first measurement portion and the second measurement portion.

13. The measurement apparatus according to claim 12, configured to estimate the state of the subject based on the information measured by the at least one of the first measurement portion and the second measurement portion and satisfying a predetermined condition.

14. The measurement apparatus according to claim 12, configured to estimate the state of the subject by taking into account at least one of time information and position information.

15. The measurement apparatus according to claim 4, configured to perform measurement by at least one of the first measurement portion and the second measurement portion while the sound output interface is outputting predetermined sound.

16. The measurement apparatus according to claim 15, configured to store a result of the measurement by the at least one of the first measurement portion and the second measurement portion while the sound output interface is outputting the predetermined sound.

17. The measurement apparatus according to claim 15, configured to change the sound output by the sound output interface, based on information measured by the at least one of the first measurement portion and the second measurement portion.

18. The measurement apparatus according to claim 1, further comprising
a communication interface configured to transmit and receive information.

19. The measurement apparatus according to claim 1, wherein the first measurement portion is configured to be worn at an earlobe of the subject.

20. The measurement apparatus according to claim 1, wherein the second measurement portion is configured to be in contact with a mastoid part of the subject in a state in which the wearing portion is worn.

21. The measurement apparatus according to claim 20, wherein the second measurement portion is configured to be biased toward the mastoid part in a state in which the wearing portion is worn.

22. A measurement apparatus comprising:
a wearing portion configured to be worn at an auricle of a subject;
a body portion including a connector configured to removably connect a measurement instrument capable of measuring biological information of the subject; and
a body temperature measurement portion joined to the body portion, and configured to be in contact with the subject in a state in which the wearing portion is worn and measure body temperature.

23. A measurement system comprising:
a measurement apparatus including: a wearing portion configured to be worn at an auricle of a subj ect; a body portion including a connector; and a body temperature measurement portion joined to the body portion, and configured to be in contact with the subject in a state in which the wearing portion is worn and measure body temperature; and
a measurement instrument configured to be removably connected to the connector, and measure biological information of the subject.
